(19) United States
(12) Patent Application Publication
(10) Pub. No.: US 2016/0151124 A1
(43) Pub. Date: Jun. 2, 2016

(54) IMPLANTABLE MARKERS

(71) Applicants: YISSUM RESEARCH DEVELOPMENT COMPANY OF THE HEBREW UNIVERSITY OF JERUSALEM LTD., Givat Ram, Jerusalem (IL); RAMBAM HEALTH CORPORATION, Haifa (IL)

(72) Inventors: Abraham J. DOMB, Efrat (IL); Zahava GALLIMIDI, Haifa (IL)

(73) Assignees: YISSUM RESEARCH DEVELOPMENT COMPANY OF THE HEBREW UNIVERSITY OF JERUSALEM LTD., Jerusalem (IL); RAMBAM HEALTH CORPORATION, Haifa (IL)

(21) Appl. No.: 14/904,198

(22) PCT Filed: Jul. 10, 2014
(86) PCT No.: PCT/IL14/50625

§ 371 Date: Jan. 11, 2016

Related U.S. Application Data
(60) Provisional application No. 61/845,127, filed on Jul. 11, 2013.

(51) Int. Cl. A61B 90/00; A61K 49/18; A61K 49/22; A61K 49/04

(52) U.S. Cl. CPC A61B 90/39; A61K 49/04; A61K 49/1803; A61K 49/226; A61B 2090/3908; A61B 2090/392; A61B 2090/3925; A61B 2090/3954; A61B 2090/3966; A61B 2090/3995

(57) ABSTRACT

The invention generally provides implants comprising polymers and contrast agents for marking and monitoring medical conditions.

### TECHNOLOGICAL FIELD

The invention generally provides implants comprising polymers and contrast agents for marking and monitoring medical conditions.

### BACKGROUND OF THE INVENTION

Breast cancer is the most common cancer in women worldwide and the principle cause of death from cancer among women globally [1]. Breast cancer is also the most common cancer of women in the United States, and the second leading cause of cancer-related mortality [2].

Several clinical studies have shown that screening women using mammography significantly reduces the risk of breast cancer mortality [3, 4]. The beneficial effects of screening are greater in older women, and become more notable when patients are followed-up over longer periods of time.

Mammography is the only method of screening for breast cancer shown to decrease mortality. Annual screening mammography is recommended starting at: 1) age 40 for general population; 2) age 25-30 for BRCA (BReast CAncer 1) carriers and untested relatives of BRCA carriers; 3) age 25-30 or 10 years earlier than the age of the affected relative at diagnosis (whichever is later) for women with a first-degree relative with premenopausal breast cancer or for women with a lifetime risk of breast cancer ≧20% on the basis of family history; 4) 8 years after radiation therapy but not before age 25 for women who received mantle radiation between the ages of 10-30; and 5) any age for women with biopsy-proven lobular neoplasia, atypical ductal hyperplasia (ADH), ductal carcinoma in situ (DCIS), or invasive breast cancer.

Breast magnetic resonance imaging (MRI) in high-risk women has been shown to have a higher sensitivity than mammography, and the combination of mammography and MRI in this population has the highest sensitivity. In a high-risk population, the combination of MRI and mammography has a higher sensitivity (92.7%) than ultrasound and mammography combined (52%). Therefore, in high-risk women for whom supplemental screening is indicated, MRI is recommended when possible.

Screening ultrasound is indicated in high-risk patients who cannot tolerate MRI. Supplemental screening with ultrasound for women with intermediate risk and dense breasts is an option. However, hand-held US screening by the radiologist has a high false-positive rate and is time-consuming Therefore, this is likely not a cost-effective practice.

There is insufficient evidence to support the use of other imaging modalities such as thermography, breast specific gamma imaging (BSGI), positron emission mammography (PEM), or optical imaging for breast cancer screening. Radiation dose from BSGI and PEM are 15-30 times higher than the dose of a digital mammogram, and they are not indicated for screening in their present form.

For high-risk women, annual screening mammography and contrast-enhanced MRI are both indicated while US can be used for patients with contraindications to MRI. For intermediate-risk women, annual screening mammography is indicated. Contrast-enhanced MRI may be indicated in some patients. For average-risk women, annual screening mammography is indicated

Although mammography is important for the early recognition of disease, less than 1% of patients with a suspicious mammogram actually have cancer. Therefore, most abnormal mammograms are false-positive findings that require additional evaluation [5].

The objective of a breast biopsy is to obtain tissue for microscopic evaluation from a suspicious breast lesion. Although very small lesions may be completely removed in some cases as a result of the biopsy procedure, the removal of suspected cancer is not the objective of the biopsy. Examination of biopsy samples by a pathologist is essential in diagnosing suspicious breast masses, determining how far the patient's cancer has advanced, and deciding on surgery and the course of treatment.

Breast needle biopsy is guided to the lesion using one of either modalities: mammography, ultrasound or MRI. The modality used for guidance selected according to achieve the best lesion visualization. Breast surgery is performed when biopsy is proved to be malignant. When biopsy is benign follow-up is continued. 20-30% of breast needle biopsy is malignant.

When the breast lesion is big enough there is no problem in detecting the lesion site (after breast needle biopsy) and marking it for surgery. When the lesion is very small the whole lesion sometimes removed during biopsy making it impossible to mark it after wise for surgery. In order to overcome this obstacle in small lesions, a metallic clip is often inserted at the site of the biopsy during the procedure. The clip is visualized by mammography and can be located and marked before surgery. Clips are inserted in all cases of MRI biopsies and in small lesions in mammography and ultrasound biopsies. The clips commonly used are metallic and are not bio degradable. Some clips include a water soluble polyethylene glycol based hydrogel polymer (which is important in ultrasound detection) known as Hydromark®.

Placement of surgical clips in the excision site is accepted as the gold standard for localizing irradiation. However, the placement of such clips consumes operative time, is not routinely performed by all surgeons, and does not completely define the excision cavity edge in three dimensions. In the absence of such clips, other techniques suggested to improve location. Presently, following biopsy a metal marking clip is placed in the biopsy cavity allowing for wire localization of the biopsy site if surgical lesion resection becomes necessary.

In the case of benign lesions the clip remains in the breast. The idea of having a foreign body in the breast does not appeal to many women especially when the result of biopsy is benign. Some women even refuse to have the biopsy knowing that a clip will be inserted. When several biopsies are performed through the years there may be several clips in the breast which can be removed only by surgery. In case of a benign result of image guided breast biopsy, a follow-up is recommended in a period of 6 months followed by a once a year routine follow-up.

Implantable biodegradable polymers have been used as temporary devices such as bone fixation plates, nails and wires, absorbable sutures and extended release drug carriers Implantable drug delivery systems combined with biodegradable polymers have attracted a great deal attention, especially after approval of Gliadel® to treat brain cancer [6]. Biodegradable polyanhydrides and polyesters have proven to be useful materials for preparing implantable controlled drug delivery devices. These implants can be injected into specific anatomic sites, providing high local drug concentrations, but low systemic levels, while minimizing adverse effects.

U.S. Pat. No. 6,350,244 [7] discloses a bio-absorbable marker that is positioned near a lesion or tumor during a biopsy procedure. The marker includes a contrast agent and is bioabsorbed slowly so that the biopsy site can be located weeks or even months later if needed,

US. 20090149746 [8] describes a two component alginate gelling material that form a hydrogel upon injection into a soft tissue biopsy cavity.

U.S. Pat. No. 8,064,987 [9] describes tissue markers of specific dual shape construction where flat films are connected with rods that pass through holes made in the films.

### REFERENCES

[1] Global cancer statistic Dr. D. Max Parkin MD1, Dr. Paola Pisani PhD2, Mr. J. erlay3, CA: A Cancer Journal for Clinicians, Volume 49, Issue 1, pages 33-64, January/February 1999.

[2] American Cancer Society. Cancer Facts and Figures 2011-2012. Atlanta, Ga.: American Cancer Society, Inc. Available at: http://www.cancer.org/acs/groups/content/@epidemiologysurveilance/documents/document/acspc-030975.pdf. Accessed Nov. 7, s2011.

[3] National Cancer Institute. Breast cancer screening. Available at: http://www.cancer.gov/cancertopics/pdq/screening/breast/healthprofessional. Updated Jul. 27, 2011. Accessed Oct. 27, 2011.

[4] American Cancer Society. American Cancer Society recommendations for early breast cancer detection in women without breast symptoms. Available at: http://www.cancer.org/Cancer/BreastCancer/MoreInformation/BreastCancerEarlyDetection/breast-cancer-early-detection-acs-recs. Accessed Oct. 27, 2011.

[5] Tabar L, Vitak B, Chen TH, et al. Swedish two-county trial: impact of mammographic screening on breast cancer mortality during 3 decades. Radiology, 2011; 260:658 p663.

[6] Domb A., Gliadel—a preparation for the supplementary treatment of brain cancer, Harefuah 1999;137(3-4):127-131.

[7] U.S. Pat. No. 6,350,244.

[8] U.S. Patent application no. 20090149746.

[9] U.S. Pat. No. 8,064,987.

### SUMMARY OF THE INVENTION

The ability to monitor a biodegradable implant will allow the precise location and temporal changes in treated areas in order to maximize patient's benefit especially when the polymer is injected in sensitive regions such as near nerves, blood vessels or other sensitive tissues. Furthermore, the visible biodegradable polymer can be used for diagnostics such as targeting of nanoparticles with surface ligands that bind to specific tissues such as cancerous tissue with or without drug loading.

In the technology being the subject of the present invention, a contrast agent such as Lipiodol was incorporated into a pasty polymer for visualization by CT, to enable determination of location of an implant in a tissue, the shape of the polymer implant, and the change upon polymer degradation. This development paves the way for novel noninvasive methods for evaluating the size of a polymer-agent complex injected subcutaneously and to assess temporal changes at the injection site. Typically, the implant or device of the invention is inserted during a surgical procedure and does not require further surgical manipulation for its excision from the body.

In order to avoid or minimize irritation and potentially inflammation and scarring by the foreign implant or device, e.g., metal clip or location wire inserted into a tissue to mark the site of biopsy, the implant or device is selected of a material which harmlessly degrades over time and avoids the need for an additional removal surgery procedure and consequently reduces the treatment and rehabilitation time period.

Thus, to avoid the presence of a foreign body (e.g. metallic marker) in the tissues following a medical procedure that requires the insertion of an implant to mark and/or monitor a medical condition, the inventors of the present invention developed a novel implant comprising a polymer and contrast agent that can biodegrade in the body over time and that can be used to monitor, mark and locate a medical condition in the body.

Thus, in one of its aspects, the present invention provides an implant comprising a contrast agent and a slowly degradable polymer, wherein the contrast agent and the polymer are in a form selected to prevent leaching of said contrast agent from said implant, the implant having a predetermined structure identifiable, e.g., by imaging, following delivery of said implant into a living tissue.

As use herein, the term “implant” refers to a bio-compatible device which may be inserted into a human or animal body, to be positioned in a tissue or organ in such away that the implant does not affect in any way the tissue or organ in which it is implanted. The implant according to the invention comprises a contrast agent and a slowly-degradable polymer that can be fabricated into any shape suitable for delivery (e.g. by injection, cannulation, catheterization or needle insertion) into the tissue or organ of the subject (e.g. via a biopsy cavity) and which maintains its structure for a defined period of time in the tissue or organ, so as to enable imaging of the implant by a suitable imaging method (e.g. computed tomography (CT), magnetic resonance imaging (MRI), mammography).

In accordance with the invention, the implant is used to mark and/or locate a site in the body (e.g. breast tissue) for use in monitoring, identification and/or diagnosis of a medical condition (e.g. growth of a tumor, disease progression, healing process). As the skilled artesian would understand, the implant is fabricated to be stable and steady and not migrate after positioning. In addition, in accordance with the invention, the implant does not trigger a local adverse reaction such as calcification/fat necrosis/granuloma formation. The device is selected to have a shape that does not imitate anatomic structures such that the implant is distinctive and easily recognized (e.g. by shape, degree of contrast) within the body and not confused with anatomical structures.

Thus, for example, an implant may be delivered into a breast lesion to mark the lesion using mammography guidance. In accordance with the invention, the implant may be in any physical form suitable for delivery into a tissue depending on the requirements of the specific medical condition and on consideration of the surgeon handling the case.

In some embodiments, the implant is in the form of a paste.

In some embodiments, the implant is in the form of a solid.

In some embodiments, the implant is in the form of a fluid that is susceptible to gelling when subjected to certain environmental conditions.

In some embodiments, the implant is in the form of a gel.

In some embodiments, the implant is in the form of micro-bubbles or air encapsulated particles. In accordance with such embodiments, the implant of the invention possess entrapped air or gas bubbles, obtained e.g., by dispersing of hollow particles within the implant continuous polymer or by forming a foam that is sealed by a top coating that creates the hollow space with in the foam.

As the versed artesian would understand gelling compositions are liquid upon injection into the body and are made to solidify upon localization at a desired site in the body. In accordance with such embodiments, the implant may comprise a two-component solution, wherein a first component is a polymerizable or crosslinkable compound and a second component is a crosslinking or polymerization agent that is mixed with the first component. The implant may be in the form of a fluid (a gelling composition) which may also comprise a pasty polymer which gels upon contact with water and increase viscosity.

In some embodiments, the gelling composition is a heat sensitive polymer (e.g. polyethylene glycol-polylactic acid (PLA-PEG) block polymer) such that it is soluble at low temperatures and precipitates at the body temperature.

In other embodiments, the gelling composition is a solution of lactide-based polymers in a water-soluble organic solvent (e.g. N-methyl pyrrolidone, NMP), which upon contact with an aqueous medium is extracted leaving the polymer to precipitate. Some non-limiting examples of gelling compositions which can be used in accordance with the invention are described in: Chitkara D, Shikanov A, Kumar N, Domb AJ. Biodegradable injectable in situ depot-forming drug delivery systems. Macromolecular Bioscience 2006;6(12):977-990; Pawar, R. P., Shinde, N. R., Andurkar, N. M., Dake, S. A., Domb, A. J., Injectable polymers for regional drug delivery From Targeted Delivery of Small and Macromolecular Drugs (Edited By:Narang, Ajit S.; Mahato, Ram I), 2010, 457-480.

In some embodiments, the implant is maintained in a fluid form during delivery into the body and solidifies into a predetermined shape and form upon localization in situ (e.g. in a breast lesion). In some embodiments, the implant is condensed at the time of delivery and expands or changes shape in the deposition site. In some embodiments, the implant is in a pasty injectable form that solidifies or increases its viscosity when deposited in the tissue. In some embodiments, the implant is a solution in an aqueous medium or N-methyl pyrrolidone (NMP) that precipitates upon change in temperature, pH, ionic strength, chemical reaction or physical interaction.

The implant comprising a contrast agent and a polymer can be delivered into the tissue by any suitable manner As the versed artesian would understand, the delivery of the implant into the tissues is carried out using minimal invasion procedures (e.g. needles) and depends on various parameters associated with the specific requirements of medical condition that is monitored, marked or diagnosed using the implant (e.g. the disease progression). For example, when the implant is used as a breast biopsy marker it may be delivered using a (e.g. flexible) tube that is guided into the tissue (e.g. breast lesion) using a suitable imaging method such as MRI. In some embodiments, the tube is coated with the non-degradable polymer and the lumen of said tune is filled with the contrast agent.

The implant may be delivered into a lesion using any device suitable for delivery of a paste, solid, gel or fluid into the body. Some non-limiting examples of devices for insertion of the implant of the present into the body are a rod, tube, a needle, a cannula, a needle, a folded balloon, a sponge or a self-expanding foam, a capsule, a surgical clip, a plate, a stent, a tube, a wire, a ring, a loop, a comb-like shape, a thin filament or a thin film, a non-tube applicator (e.g., wherein the device is a rod wherein the implant is at the distal part of the rod and is released at the site using a mechanical, chemical or electronic trigger) and a trocar.

Thus, for example, the implant may be in a shape of an elastically compressible tube (e.g. a cannula or needle having a lumen) having a distal end portion and a proximal end portion, adapted to extend through the body and to be guided to the selected tissue location (e.g. breast) using an imaging system (e.g. CT, MRI, ultrasound guidance).

In some embodiments, the implant is in a form of a compressible/self expanding sponge or foam suitable for delivery into the body using a dry hydrogel such as hyaluronic acid that upon contact with body fluids swells and expands into a hydrogel.

In some embodiments, the implant is in a form of a compressible/self expanding sponge or foam that is formed through a process of carbon dioxide foaming, wherein sodium carbonate and citric acid are mixed in a polymer or a dry hydrogel and upon contact with body fluids form a CO₂ gas which expands the polymer.

In some embodiments, the implant is fabricated into a matrix form wherein the contrast agent is uniformly dispersed in the polymer as a carrier to form a matrix. In accordance with such embodiments, the implant may be prepared by a process of either melting, dissolution or sintering wherein the contrast agent is either added to a polymer melt or where the contrast agent melts at a temperature above the melting point of the polymer, it can be melt mixed and injection molded into the desired device shape and size.

In a typical procedure, the polymer is melted and at least one contrast agent, which may be in any form, e.g., in fine powder form, (such contrast agents in fine powder form may be iron oxide and/or barium sulfate and/or gadolinium sulfate) is added to the polymer melt at a concentration of between about 1% to 5% (contrast agent loading) to form a uniform mixture which is subsequently extruded as thin filament or molded into a desired shape.

Alternatively, the polymer may be dissolved in a solvent, such as ethyl acetate, to form a viscous solution, where one or more contrast agents are mixed in and molded into filaments or other shapes. In case of sintering, a homogeneous powder mixture of the polymer and one or more contrast agents is compressed under a certain temperature to form an implant according to the invention.

In some embodiments, the implant is fabricated into a form of a capsule or envelope, wherein the contrast agent may be contained within an envelope or coating made of the polymer. In such embodiments, a contrast agent is first embedded at a high concentration within a polymer filament or polymer structure, and subsequently the filament or structure are coated with a film, a coat or a layer of pure polymer or of a polymer mixture comprising a low concentration of the contrast agent. This coating ensures protection of the device from early release of contrast agent or drastic change in device shape.

As the skilled artesian would understand the need for encapsulation exists when the contrast agent (e.g. Lipiodol) is a liquid at room temperature. In such a case, the contrast agent (e.g. Lipiodol) is absorbed in a polymer sponge or encapsulated into particles or hollow fibers that can be incorporated in another carrier, such as a biodegradable polymer, that maintains its shape for a limited time period and biodegrades thereafter.

In some embodiments, the implant is a pasty hydrophophic polyester made from the polycondensation of hydroxy acids and castor oil.

In some embodiments, the implant is made of lactic acid and glycolic acid co-polymers and block co-polymers with polyethylene glycol that are soluble in a pharmaceutically acceptable solvent such as buffer solution, water or N-methyl pyrrolidone.

In some embodiments, the implant is made of lactic acid/glycolic acid/caprolactone polymeric materials that alter their shape upon insertion into tissue due to change in body temperature, pH and ionic strength or by external irradiation.

In some embodiments, the implant is a hybrid combination of hydrogel and hydrophobic biodegradable polymers (e.g. poly-L-lactide, poly(lactic-co-glycolic acid and polycaprolactone) in combination with cross-linked polysaccharide or gelatin or a high molecular weight polysaccharide such as hyaluronic acid, chitosan and arabinogalactan.

In some embodiments, the implant is a poly-L-lactide rod coated with a hydrogel.

In some embodiments, the implant is a biodegradable polymer non-isotropically loaded with gas generating agents that upon reaction with water or upon a temperature change, releases gas that either inflates the polymer sample or bend it and endow it with a different shape.

In some embodiments, the implant is a folded balloon that is inflated by filling with saline. The folded balloon may contain a dry gel that absorbs water from the surrounding tissue that inflates the balloon.

In some embodiments, the implant is in the form of a sponge or a self-expanding foam that can be modified by the practitioner (e.g. by trimming) to suit the shape requirement for delivery into a specific tissue (e.g. breast tissue).

In some embodiments, the implant can be used as a capsule for site specific delivery of drugs.

In some embodiments, the implant can be used as a surgical clip in surgical procedures.

In some embodiments, the implant can be used as a plate to aid healing fractures.

In some embodiments, the implant can be used as a stent (e.g. a coronary stent, a vascular stent).

In accordance with the present invention, the implant may be of any form. Where the implant is in the form of a tube or a wire or generally is charactarizable by at least one long axis, the size (length) of the long axis may be between about 2 mm and about 10 millimeters. The short axis, or the width or thickness of the implant, may be between about 0.1 and between about 2 mm

In some embodiments, the implant further comprises one or more components selected from plasticizers (e.g., tributyl citrate, polyethylene glycol or fatty acid esters), flow promoters, polymer processing aids, gelling initiators, viscosity modifiers, anti-microbial agents and combinations thereof.

In accordance with the present invention, the implant may be visualized by any suitable imaging method. Some non-limiting imaging methods suitable for visualizing the implant of the present invention include X-ray radiography mammography, positron emission tomography-computed tomography (PET-CT), ultrasound, MRI, CT, endoscopy, elastography, tactile imaging, thermography, medical photography, nuclear medicine and functional imaging techniques as positron emission tomography.

Thus, for example, the implant can distinguish between same and separate lesions in two different modalities, e.g., determine the location of a lymph node in the groin/axilla with uptake in PET-CT and further viewing the same lymph node by ultrasound.

As used herein, the term “contrast agent” refers to a medical contrast medium being a substance used to enhance the contrast of structures or fluids within the body in medical imaging (e.g. MRI, CT). As the versed artesian would understand, the herein described contrast agent is an agent which is clinically safe, inert and stable under physiological conditions when incorporated into the implant of the invention together with the polymer.

In some embodiments, the contrast agent of the invention is in a form selected from micro- and nanocapsules, iron oxide magnetic nanopaticles, magnetic particles, gold nanoparticles, safe mineralized particles, microspheres and metal oxides or sulfates that are loaded with the contrast agent of the invention. In accordance with the present invention, the contrast agent may be dispersed, complexed or chemically bound to the polymer carrier. In some embodiments, the contrast agent may be chemically associated with a polymer, e.g., a co-polyester of Lipiodol with citric acid or fatty diacids or a polycarbonate, when reacted with phosgene.

In other embodiments, the contrast agent is attached to the end of the polymer.

In some embodiments, the contrast agent is in the form of a physical mixture with the polymer. In accordance with such embodiments, the contrast agent may be dissolved, dispersed or emulsified in the polymer carrier or sintered with the polymer powder. In some embodiments, the contrast agent is a co-monomer incorporated (e.g. together with other monomers) into the implant of the invention.

In some embodiments, the contrast agent is contained in at least one monomer unit within the polymer. In accordance with such embodiments, the Lipiodol and iodinated contrast agents, which have functional groups such as alcohols, carboxyalic acids and amines, can be polymerized or copolymerized with an hydrolizable bond and may be contained in at least one monomer unit within the polymer.

In some embodiments, the contrast agent is encapsulated or entrapped using a process of e.g., microencapsulation or microsphere preparation within the polymer.

Some non-limiting examples for contrast agents that can be attached to or associated with a polymer for forming an implant according to the invention are:
iohexol that contain 6 hydroxy groups and is used as starting alcohol for the polymerization of lactide, glycolide or caprolactone and their copolymers.
the polyester or polyanhydride copolymrization of carboxylic acids of iodo contrast agents or gallium complexes with carboxylic acid containing complexes.

Some non-limiting examples of contrast agents that can be used in accordance with the present invention are X-ray and computed tomography (CT) water soluble iodinated contrast agents (e.g. Diatrizoic acid, Metrizoic acid, Iodamide, Iotalamic acid, Ioxitalamic acid, Ioglicic acid, Acetrizoic acid, Iocarmic acid, Methiodal and Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid and Calcium iopodate); Hydrophobic iodinated contrast agents (e.g. Ethyl esters of iodised fatty, acids, Iopydol, Propyliodone, Iofendylate, Lipiodol and non-iodinated salt, barium sulfate); Magnetic Resonance Imaging (MRI) contrast agents (e.g. Gadolinium-based: Gadobenic acid, Gadobutrol, Gadodiamide, Gadofosveset, Gadolinium, Gadopentetic acid, Gadoteric acid, Gadoteridol, Gadoversetamide, Gadoxetic acid, gadolinium oxide, carbonate, chloride, bromide fluoride, sulfates and other gadolinium salts and gadolinium complexes with organic and inorganic molecules; Iron oxide and salts and magnetic iron derivatives); ultrasound contrast agents (e.g. Microspheres of human albumin Microparticles of galactose, Perflenapent, Microspheres of phospholipids, Sulfur hexafluoride and air entrapped bubbles); short half-life radioactive agents (e.g. technetium and low hazard radioactive containing molecules such as tritiated molecules).

As used herein, the term “slowly-degradable polymer” or any lingual variation thereof, refers to a polymer or mixtures of polymers that can be delivered (as part of the implant of the invention) to a tissue by any method described herein and that is fabricated to maintain its physical form in the tissue (e.g. breast lesion) for a predetermined time period (e.g. a few weeks to a few months). In some embodiments, the polymer loses 1% of its weight after 10 days. In other embodiments, the polymer is selected to lose at most 2, 3, 4, 5, 6, 7, 8, 9, or 10% of its weight after 10 days from the time of implantation.

In other embodiments, the polymer is selected to lose between 1 and 5% of its weight within 10, 20, 30, 40 50, 60, 70, 80, 90 or 100 days following time of implantation.

In further embodiments, the polymer is selected to completely degrade in the site of implantation within a period of 1, 2, 3, 4, 5, 6, 7, 8, 9, 10 or more weeks, or within a period of 1, 2, 3, 4, 5, 6, 7, 8, 9, 10 months, or within a period of 1, 2, 3, 4, 5, 6, 7, 8, 9, or 10 years.

In accordance with the invention, the polymer may be a homopolymer, a copolymer or oligomer (including dimers) and may have a linear, branched or cyclic structure. The polymer employed in accordance with the invention is in a form selected to prevent leaching of the contrast agent from the implant. Thus, in accordance with the invention, the polymer and contrast agent that constitute the implant are mixed together to form an implant that is fabricated for delivery into a tissue, such that the contrast agent is maintained within the implant together with the polymer without leaching of the contrast agent to the surrounding tissue and thus enable the practitioner to distinguish the tissue carrying the implant (e.g. breast lesion) from neighboring tissue by a suitable imaging method, as described herein.

Thus, for example, when the implant is fabricated into a form of a matrix the contrast agent dispersed in the matrix does not leach out from the matrix during the life time of the implant, as described herein. Accordingly, the implant is fabricated to maintain a predetermined structure identifiable (e.g. by MRI, CT, mammography or any suitable imaging system) following delivery of said implant into a living tissue.

The implant of the present invention is biocompatible, physiologically tolerated under physiological conditions and stable under (e.g. human) body temperature does not biodegrade in the tissue for a defined time period. In some embodiments, the implant is fabricated to biodegrade in the body into nontoxic material after a time period as defined above. As used herein, the term “biodegrade” generally refers to a biologically assisted degradation process that the polymer making-up the implant undergoes in a biological environment, such as within the body of a patient. As the versed artesian would understand, biodegradation encompasses within its scope the processes of absorption, dissolution, breaking down, degradation, assimilation, or otherwise removal of the implant from the body, a biological environment. The biodegradation is complete when the implant is no longer detectable within the body by imaging (e.g. CT, ultrasound).

The implant should typically remain in place (e.g. breast lesion) and be detectable (e.g., by CT, MRI) within the tissue for a period of at least one month.

In some embodiments, the implant is fabricated to biodegrade spontaneously after a defined time period. In accordance with such embodiments, the implant contains a polymer or a mixture of polymers that spontaneously degrade in the body after a defined time period, as defined herein, that is dictated by the type of polymer(s) and/or the ratio of polymers in the mixture and/or by the form of the implant (e.g. self expanding sponge, flexible needle etc). As the person versed in the art would understand, biodegradable polymers for preparing the implant of the invention are selected based on the required residency time of the implant in the tissue, its ability to maintain its shape in the body and provide clinical data via imaging, as described herein, to the practitioner.

Accordingly, for periods of one to several weeks polyester-anhydrides, poly(lactide-glycolide) or a natural polymer are suitable. For periods of several months, PLA, PCL and polyethylene carbonate homo and copolymers or blends are suitable.

In some embodiments, the implant is fabricated to biodegrade following an external stimulus or stimuli. In accordance with such embodiments, the polymer may be a stimulus-responsive polymer that starts to biodegrade following an external stimulus such as an electrical current; magnetic field; change in temperature or pH; irradiation.

Thus, the implant of the present invention is designed to degrade at different time frames, depending on its chemical composition and on the requirements dictated by the medical condition or by any other parameters recognized by the skilled artesian. For example, an implant comprising polyesters made from lactic, glycolic, hydroxybutyric and hydroxyl caprilic acid may degrade in vivo for periods from a few weeks to about two years and an implant comprising polanhydrides, oxidized cellulose or gelatin may be degraded and eliminated from the body within days to weeks.

In some embodiments, the polymer is a shape memory polymer being a material that has the ability to return from a deformed state (temporary shape) to its original (permanent) shape induced by an external stimulus, such as temperature change and to be designed with an optimum biodegradability and with adjusted recovery temperatures depending on the selection of the copolymer composition as understood by the person versed in the art. Some non-limiting examples of biodegradable shape memory polymers include PCL-PLA multi block copolymers, PCL-polyurethane block copolymers.

In some embodiments, the (e.g. stimulus-responsive) polymer in the implant encapsulates or entraps the contrast agent (e.g. in a form of a tube or rod, ring, loop, comb-like shape, thin filament, thin film) such that the outer portion of the implant is made of the polymer and following degradation of the polymer the contrast agent is non-toxically released into the surrounding tissue to mark the biodegradation of the implant. The biodegradation characteristics (e.g. rate) depend on many factors including the micro and molecular structure of the implant, the ratio of copolymer and/or homopolymers in the implant, the shape of the implant and the implantation site (e.g. breast tissue), etc.

Some non-limiting examples of polymers that can be used in the implant of the present invention polyethylene glycol-polylactic acid (PLA-PEG) block polymer, poly(sebacic-co-ricinoleic acid), Poly(lactic acid) (PLA), polycaprolactone (PCL), polyethylene carbonate homo and copolymers or mixtures thereof; PCL-PLA, PCL-PLA multi block copolymer, PCL-polyurethane block copolymer; polyesters made from lactic, glycolic, hydroxybutyric and hydroxyl caprilic acid; polyester-anhydrides, polanhydrides, oxidized cellulose or gelatin; Poly(L-lactic acid) (L-PLA), poly(lactic-co-glycolic acid (PLGA) copolymer, polydioxanone (PDS), polyglycolide (PGA), poly(lactide-glycolide), poly(glycolic acid)/Tri-Methylene Carbonate (TMC), chitosan, cellulose, amylose, gelatin, collagen, hyaluronic acid, dextran and any derivatives or mixtures thereof.

In another one of its aspects the present invention provides, a method of placing an implant in a tissue or organ in vivo, the method comprising:
implanting, the implant of the invention through an incision made in a tissue or an organ of a subject (the incision may be made for the purpose of inserting the implant or may be done for surgical purposes); and
guiding the implant into the desired tissue or organ site in vivo.

In some embodiments, the method further comprises visualizing the tissue or organ prior to, during or after insertion in order to correctly position the implant in the tissue or organ. The visualization may be achieved using one or more imaging techniques as described herein.

In still another one of its aspects the present invention provides, a method of imaging a tissue having been inserted an implant according to the invention, the method comprising imaging a tissue or an organ having been inserted with an implant of the invention to identify at least one of position, shape, constitution and any other factor which may be indicative of the implant state or its surrounding.

The method may also be used for monitoring, marking or locating a medical condition in the body.

The present invention also provides a process for the preparation of the implant of the invention, the process comprising:
mixing at least one contrast agent with at least one polymer;
melting the mixture of at least one contrast agent and at least one polymer to obtain a homogenous mixture;
forming the implant having a desired shape and size.

The forming of the implant may be achieved by molding, extruding or otherwise mechanically achieving the desired shape and size.

In some embodiments, the contrast agent is iron oxide and the polymer is a mixture of PLA/PCL:LA.

In some embodiments, the contrast agent Lipiodol and the polymer is poly(sebacic-co-ricinoleic acid).

In some embodiments, the process for the preparation of the implant of the invention comprises dissolving at least one contrast agent with at least one polymer to obtain a homogenous and viscous mixture following evaporation of the solvent.

In some embodiments, the contrast agent is a Gadolinium complex, the polymer is a mixture of PLA/PCL:LA and the solvent is Dichloromethane (DCM).

In other embodiment, the process for the preparation of the implant comprises:
absorbing at least one contrast agent is a polymeric sponge;
coating the sponge with a solution of at least one polymer;
drying the sponge at room temperature.

In some embodiments, the sponge is a polyurethane sponge, the contrast agent is Lipiodol and the polymer solution is PCL:LA in DCM.

### DETAILED DESCRIPTION OF EMBODIMENTS

### Methods

Pasty polymer lipiodol formulation

To noninvasively evaluate the visibility, shape, and degradation of an implant containing Lipiodol (an x-ray contrast medium) in vitro using CT, Lipiodol was incorporated in poly(sebacic-co-ricinoleic acid) P(SA:RA), a biodegradable injectable pasty polymer, and CT visibility was assessed. For ex vivo evaluation, bovine liver was injected with the polymer loaded Lipiodol; on the other hand, for in vivo evaluation rats were injected subcutaneously with Lipiodol in polymer and CT was performed. Results obtained showed that polymer diameter at CT can be correlated with implant weight and pathological measurements. Polymer formulation containing 5% Lipiodol was visible on CT in vitro. Ex vivo tests showed a round polymer deposit at the injection site compared with free dispersion of Lipiodol alone. Correlation between implant size at CT scan and surgery at 48 hours was R²=0.78. Average CT diameter at 9 days was 14.2±2.8 mm, as compared to 7.3±1.1 mm in controls. After 9 days, the implant degraded into several zones containing inflammatory cells seen on CT with increased heterogeneity. In conclusion, Lipiodol incorporated in P(SA:RA) is visible on CT, and polymer degradation can be potentially monitored noninvasively.

Formulation of Lipiodol in polymer:

Poly(sebacic-co-ricinoleic ester anhydride), P(SA:RA)30:70 was synthesized by transesterification followed by anhydride melt condensation as described in the art. A pasty injectable polymer with an average molecular weight (Mw) of 5,400 Da and a melting temperature of about 30° C. was used in this study. Lipiodol with 38% iodine by weight was purchased from Laboratories Guerbet (Aulnay-sous-Bois, France); Iopamidol, a hydrophilic contrast agent (300 mgI/ml) from Bracco, Italy, was used in the control arm (1 ml injection). Other polymer formulations that have been used as markers: pasty and solid polymers prepared from the polycondensation of castor oil and lactic acid, glycolic acid and caprolactone. Copolymers of PLA-PEG that form clear solution in water at temperatures below 20° C. and solidify at body temperature. Lipiodol was incorporated by either dispersion or solubilization in the polymer mass.

Lipiodol was incorporated into P(SA:RA) by mixing Lipiodol oil and polymer in mortar and pestle at room temperature without any solvent until a homogeneous paste was obtained. This technique allowed incorporation of sensitive drugs such as peptides and proteins, in formulation without deterioration. The polymer and the formulations remain unchanged with regard to polymer molecular weight and content of marker when stored under dry nitrogen at refrigeration (4° C.) for at least three months.

CT technique and evaluation

Studies were performed on a 64 slice MDCT (Philips Medical Systems, Cleveland Ohio). Study parameters were 120 kVp, 240 mAs, and slice thickness 1.2 mm with 0.6 mm increment. Abdominal and bone windows (used for viewing bone structure and joints in CT scanning) were used for visualization. An experienced radiologist with 10 years of experience performed measurements using electronic calipers of a dedicated workstation (EBW 4.0, Philips Medical Systems, Cleveland Ohio).

In vitro analysis

CT scans were performed to evaluate blank P(SA:RA) formulations, as well as blended polymer-Lipiodol in concentrations of 5%, 10%, 25% (w/w) and pure Lipiodol (n=3). Formulations were kept in glass bottles and tested for homogeneity. Estimates of density were based on CT analysis. Results are average of three measurements.

Ex vivo analysis

Since a 10% Lipiodol formulation had high Hounsfield unit (HU) densities in in vitro experiments, concentrations of not more than 5% were used in this experiment. Lipiodol at concentrations of 0.1%, 0.5%, 1%, and 5% (w/w) were incorporated in P(SA:RA)30:70 by trituration. Formulations and control injections of Lipiodol, blank polymer and saline were injected via a 19 gauge needle into ex vivo bovine liver with the injected materials as shown in Table 2. A CT scan was performed immediately after injection. The diameter of the mixture deposited was calculated as the average of length and width measured in the CT scan. Results are average of three measurements.

In vivo analysis

Animal Ethics Committee approval was obtained prior to the study. Lipiodol 5% in P(SA:RA) was used, since 1% or less Lipiodol concentrations are not visible with CT in the ex vivo experiment. Inbred female F344/NHsd rats, 18-21 weeks, weighing about 200 g (Harlan Sprague Dawley, Indianapolis Ind.) were kept under specific pathogen free conditions and given free access to irradiated food and acidified water throughout the experiment.

Test group rats (n=7) were anesthetized by chloral hydrate (0.64 ml/100 g body weight) and injected subcutaneously to the back with 0.5 ml of formulation containing 5% Lipiodol (w/w) in P(SA:RA) (total of 0.025g Lipiodol). Control rats (n=4) were anesthetized by chloral hydrate and injected subcutaneously to the backspace with 0.2 ml saline as a sham injection and Lipiodol without polymer (0.1 ml, a four-fold Lipiodol dose compared with the polymer formulation) was injected subcutaneously into a different site. A blank polymer was not injected, since it is not visible in CT in the ex vivo experiment. CT scans were performed immediately after the injections and on predefined time points. The precision of injection is 0.05 ml.

Three rats from the test group and three rats from the control group were re-anesthetized 48 hours after injection, and CT scan was again performed. The three test group rats were anesthetized by chloral hydrate. Their skins were sampled at the injection site, and the polymer implants were exposed, measured, and weighed (wet and dry). Nine days after injection, the remaining four test group rats and three control group rats were anesthetized, and CT scans were performed. The seven rats were anesthetized by chloral hydrate. The animal's skins were sampled at the injection site and polymer implants were exposed, measured, and weighed (wet and dry).

### Histopathology

Sample tissues from the injection site in rats were sent for histopathology evaluation. Implants were taken for chemical analysis. Surrounding tissues were fixed in 4% neutrally buffered formaldehyde and subjected to histopathological examination. Tissues were trimmed, embedded in paraffin, and routinely processed for light microscopy. Sections were stained with hematoxylin and eosin (H&E). Each sample was evaluated and graded for histopathological changes. Reactive and inflammatory changes were assigned severity grades representing unremarkable, minimal, mild, moderate, and marked changes. Samples were assessed for presence or absence of capsule and histological components of the capsule, e.g., inflammatory cells including giant cells, fibroblasts, and mature collagen. All tissues did not show significant signs of toxicity.

Statistical analysis

Descriptive statistics were used when applicable. Linear correlation was assessed for correlating sizes in CT and histopathology. Student t-test was used for differences in CT sizes of the injected polymer in polymer-Lipiodol group compared with animals injected with Lipiodol only. All analyses were performed with SAS statistical analysis software (version 9.1, SAS Institute, Inc., Cary, N.C.). p<0.05 was considered significant.

### RESULTS

### Example 1

In vitro analysis

Formulations were found to be homogeneous and stable for months. Lipiodol was highly visible in P(SA:RA) at concentrations of 5% with a density of 1866 HU (SD-150 HU.

Ex vivo analysis

Blank polymer without contrast medium was not visible in the CT scan. Lipiodol 5% in P(SA:RA) was visible at 0.2 ml and at 1 ml injection volumes. The implanted polymer attains a round shape after injection and remains at the injection site. The polymer is not visible on CT with contrast concentrations of 1% Lipiodol content or less. Control injections of Lipiodol were visible, but dispersed freely in the liver.

In vivo analysis

CT scans immediately after injection show an average polymer diameter of 10.2±1.04 mm Average diameter of the control Lipiodol deposit at immediate CT is 7.15±2.69 mm, with greater dispersion in comparison with the Lipiodol-P(SA:RA) injection (p=0.02). The Lipiodol control 0.1 ml injection is freely dispersed. CT scan performed 48 h after injection shows an average polymer diameter of 10.28±2.03 mm, compared with an average diameter of 7.38±1.67 mm with more dispersing in the control animals (p=0.01). The implant diameter does not change at 48 h, although there is some biodegradation, seen as black bubbles. Pathological evaluation conducted at 48 hours after injection shows an average estimated polymer diameter of 13.8±1 8 mm, with a wet weight of 423±46 mg and dry weight of 310±15 mg. Histopathological tolerance assessment shows a mild reaction in 2 rats anesthetized after 48 hours. (The capsule was composed of mild, proliferating fibroblasts, with an inner layer composed of mild polymorphonuclear cells, and more internally, a mild presence of necrotic material) Implant size on CT, and after anesthetization at 48 hours was found to be 10.28±2.03 mm measured in the CT scan and 13.8±1.8 mm measured at surgery (R²=0.78).

In comparison to day 2, the average polymer diameter on CT 9 days after injection increases to 14.2±2.78 mm compared with a stable average diameter 7.3±1.06 mm in the control animals, which is also evidenced with greater heterogeneity and more fluid with low density areas. Evaluation at 9 days after injection, demonstrates a wet weight of 233.75±40 mg and dry weight of 195±20 mg. After 9 days the implant evolved into several zones containing inflammatory cells, with most of the polymer degraded. On CT, polymer biodegradation was shown as black bubbles. Measurement of average HU values was not possible for the polymer-Lipiodol, since it contained both high and low density areas.

### Example 2

Inflatable device

To a viscous solution of poly-L-lactide (PLLA) in NMP, a fine powder of sodium carbonate and citric acid (1:1 w/w ration) is added and the solution is either extruded to form a filament, cast into film or allowed to dry into a cast or different shapes. The pasty composition forms bubbles with entrapped CO₂ when exposed to water where the salts react in water to generate CO₂.

The gas generating powder is incorporated in the polymer solution at certain locations and evaporated to dryness to form a rod or other shapes. Upon interaction with water the generated gas will bend the rod and change its shape.

### Example 3

Swellable biodegradable hollow fibers.

Hollow fibers, filaments or sleeves are filled with a hydrogel such as hyaluronic acid, oxidized cellulose, crosslinked gelatin, agarose and alginate salt gel. These gels are dried within the tube by lyophilization or other means to form a low diameter that can be inserted to a desired place in tissue where the implant absorbs water and expands. The tube composition may contain a contrast agent and the inner gel may contain another marker. Another option is to coat a biodegradable filament or rod with a biodegradable dry hydrogel such as hyaluronic acid or gelatin where upon exposure the water the hydrogel absorbs water and forms a hydrophilic rod with a solid inner core.

Hollow microparticles are prepared by coating of dissolvable microspheres with the desired polymer and dissolve out the inner core using a solvent that dissolve the inner material but does not affect the shell. For example, gelatin is used to coat polystyrene spheres where immersing the coated particles in solvent that leaves intact the outer shell. Expandable microspheres are prepared by coating of a dry hydrogel particle with a flexible polymerwhere upon contact with water the hydrogel absorbs water and gradually expands until full water absorption. The outer polymeric coating may break or creak.

### Example 4

Polymeric elements that change shape when placed in tissue

Biocompatible shape-memory polymers with the appropriate physical and mechanical properties are synthesized from poly(e-caprolactone) (PCL) diol and poly(L-lactide) (PLLA) diol with 4,40-(adipoyldioxy)-dicinnamic acid (CAC) dichloride as a chain extender derived from adipoyl chloride and 4-hydroxycinnamic acid. Copolymers of CAC/PCL/PLLA with a cyclic thermomechanical experiment are synthesized using the methods described in: Nagata and Sato, J. Polymer Sci. Part A: Polym. Chem. 43 (11) 2426-2439. Film strips and filaments of this copolymer photocured for 30 min showed good shape-memory properties with high recovery rate. The formation of the network structure and the crystallization and melting of the PCL segments contributed to this property.

### Example 5

Preparation of polymers containing contrast agents for visualization using radiology.

Samples were prepared from poly lactic acid P(LA) and Poly(L-lactide-co-ε-caprolactone) P(CL:LA, 70:30) were used as biodegradable polymers. Iron oxide, gadolinium complex and Lipiodol ultra fluid were used as contrast agents.

Polymers containing contrast agents were prepared by either melting of the polymer or by polymer dissolution in organic solvent followed by mixing with contrast agents followed by cooling or solvent evaporation respectively to prepare pellets using Teflon template. Appropriate method was selected according to the melting temperature of the contrast agent. Sample with 1% and 5% w/w contrast agent were prepared for each polymer. For Lipiodol, Polyurethane sponge was used as pattern to absorb the Lipiodol and then coated with polymer.

### Materials

1. Poly(L-lactide-co-ε-caprolactone) P(CL:LA) RESOMER® LC 703S B. N. R12020004 Mw=130000 Da
2. Poly(Lactic acid) P(LA) Mw=30000 Da
3. Iron(II,III) oxide, Fe₃O₄, Mw=231.53 Da, CAS Number: 1317-61-9, M.P=1538° C.
4. Diethylenetriaminepentaacetic acid gadolinium(III) dihydrogen salt hydrate (Gadopentetic acid), C₁₄H₂₀GdN₃O₁₀·xH₂O, Mw=547.57 Da, M.P=129° C.
5. LIPIODOL ULTRA FLUID, It contains ethyl esters of iodised fatty acids of poppy seed oil, 480 mg of iodine per mL. Total iodine 4.8 g per 10 mL (38% m/m).
6. Dichloromethane (DCM), CAS: 75-09-2
   1. Preparation of formulations with Iron oxide

The pellets contain Iron oxide made by melting method, were prepared as follow: 2 mg or 10 mg of Iron oxide were added to 198 or 190 mg of polymer PLA/PCL:LA (both were used) respectively and mixed with heating to 160° C. to yield 1% or 5% w/w contrast agent/formulation respectively. Polymers were well manual mixed to receive a homogenous mixture. The mixture was transferred to a Teflon template to get a pellets shape and cooled to room temperature.

2. Preparation of Polymers with Gadolinium complex

The pellets contain Gadolinium complex made by dissolution method. 2 mg or 10 mg of Gadolinium complex and 198 or 190 mg of polymer PLA/PCL:LA (both were used) respectively to yield 1% or 5% w/w contrast agent/formulation respectively. The polymer sample was dissolved in 700 μl of DCM while well manual mixed to receive a homogenous and viscuous mixture. The mixture was transferred to a Teflon template to get a pellets shape.

3. Preparation of Polyurethane sponge with Lipiodol

A small polyurethane sponge (5 mg) was used as pattern to absorb Lipiodol, were sponge sample incubated in 300 μl of Lipiodol for 20 min, a 266 mg weigh increase was found and then the sponge was coated with 20% w/v solution of PCL:LA in DCM in cubic Teflon template and dried at room temperature for overnight. The weight of top coating was found to be 330.4 mg (total 596.42 mg).

Samples for radiological visualization in X-Ray, MRI and ultrasound were prepared as summarized in the following table:

| | Sample Description | | | | | |
|---|---|---|---|---|---|---|
| Material | | Concentration | Contrast | Method of | Weight (mg) | |
| Code | Polymer | (% w/w) | agent | preparation | Sample I | Sample II |
| MHZ-2-9 A(*) | PCL:LA | 1 | Iron oxide | Melting | 46.7 | |
| MHZ-2-9 B | PCL:LA | 5 | Iron oxide | Melting | 70.11 | 60.89 |
| MHZ-2-9 C | PCL:LA | 1 | Gadolinium | Solvent | 31.88 | 30.08 |
| | | | complex | evaporation | | |
| MHZ-2-9 D | PCL:LA | 5 | Gadolinium | Solvent | 40.15 | 65.97 |
| | | | complex | evaporation | | |
| MHZ-2-9 E | PLA | 1 | Iron oxide | Melting | 57.69 | |
| MHZ-2-9 F | PLA | 5 | Iron oxide | Melting | 62.13 | |
| MHZ-2-9 G | PLA | 1 | Gadolinium | Solvent | 35.5 | 40.5 |
| | | | complex | evaporation | | |
| MHZ-2-9 H | PLA | 5 | Gadolinium | Solvent | 29.38 | 33.66 |
| | | | complex | evaporation | | |
| MHZ-2-9 I | Polyurethane sponge with LIPIODOL coated PCL:LA | | | | 596.42 | |
| MHZ-2-9 J | PCL:LA | Blank | — | Solvent | 58.17 | 72.74 |
| | | | | evaporation | | |
| MHZ-2-9 K | PLA | Blank | — | Solvent | 52.86 | 44.26 |
| | | | | evaporation | | |
| MHZ-2-9 L | PCL:LA | Blank | — | Melting | 95.76 | |
| MHZ-2-9 M | PLA | Blank | — | Melting | 96.27 | |

(*)A sample of fiber (30-40 cm length, ~30-50 μm fiber thickness) made from the same polymer - PCL:LA with 1% w/w Iron oxide was prepared.

Homogenous formulation was resulted by both mixing method, melting and solvent evaporation. No contrast agent precipitating was found. Samples with higher concentration of contrast agents such as 30% w/w were also prepared and were found chemically stable and homogenous. Samples prepared by solvent evaporation were identified with bubbles formation in the pellets. Fibers preparation contains contrast agents at different % were found achievable and also it can be controlled to result with the final desired length and diameter of the fiber and it is mechanical properties. Lipiodol was found to absorbed by sponges were for first test it was found to be absorbed by polymer sponge.

### Example 6

Synthesis of iohexol-PLA branched polymer: Iohexol (1%, 5% and 10% w/w to lactide) is mixed in melted lactide containing 0.2% w/w of stannous octoate as catalyst and the mixture was pursed with dry nitrogen, sealed and left to polymerize at 130° C. overnight. Solid polymers were obtained at >90% yield. The polymers possessed low viscosity with the lowest viscosity determined for the 10% iohexol content Similarly, copolymers with lactide: glycolide at 1:1 and 3:1 w/w ratio and copolymers with caprolactone and copolymers with lactide and glycolide are prepared.

### Example 7

Preparation of insert containing air bubbles: Sponges of biodegradable polymers are prepared by various methods as described in numerous papers (for example: Biomaterials Volume 21, Issue 24, 2529-2543). These scaffolds that contain >60% air are coated with a hydrophobic coating so that the entrapped air remain entrapped throughout the application, 6 months for mammography follow-up. Thin rods/fibers containing air bubbles are prepare by solvent drawing of the polymer containing a solvent that upon heat the fibers at the boiling of the co-solvent that is entrapped in the fibers form bubbles with the polymer rod.

Alternatively, air bubble capsules that are in clinical use are incorporated in the polymer that upon casting of the device, the air bubble capsules are evenly distributed within the polymer matrix.

1-37. (canceled)

38. An implant in a pasty or solid form, the implant comprising a contrast agent and a hydrophobic fully biodegradable polymer,
wherein the contrast agent and the polymer are in a form selected to prevent leaching of said contrast agent from said implant, the implant having a predetermined structure identifiable by at least one imaging method selected from computed tomography (CT), magnetic resonance imaging (MRI), ultrasound and mammography, following delivery of said implant into a living tissue, and
wherein the contrast agent is in an amount that is at least 10% of the weight of the implant.

39. The implant according to claim 38, for use in monitoring, identification and/or diagnosis of a medical condition.

40. The implant according to claim 39, wherein the medical condition is a breast lesion.

41. The implant according to claim 38, wherein the polymer is a heat sensitive polymer that is soluble at low temperature and precipitates at body temperature.

42. The implant according to claim 41, wherein the heat sensitive polymer is polyethylene glycol-polylactic acid (PLA-PEG) block polymer.

43. The implant according to claim 38, wherein said implant is maintained in fluid form during delivery into the body and solidifies into a predetermined shape upon localization in situ.

44. The implant according to claim 38, wherein said implant is condensed at time of delivery and expands or changes shape in the deposition site in tissue.

45. The implant according to claim 38, wherein said implant is in pasty injectable form that solidifies or increases its viscosity when deposited in the tissue.

46. The implant of claim 38, wherein said implant is fabricated to biodegrade spontaneously after a defined time period.

47. The implant according to claim 38, wherein said implant is fabricated to biodegrade following an external stimulus.

48. The implant according to claim 47, wherein the external stimulus is selected from an electrical current; magnetic field; change in temperature or pH; and irradiation.

49. The implant according to claim 38, wherein the polymer is selected from the group consisting of polyethylene glycol-polylactic acid (PLA-PEG) block polymer, poly(sebacic-co-ricinoleic acid), Poly(lactic acid) (PLA), polycaprolactone (PCL), polyethylene carbonate homo and copolymers or mixtures thereof; PCL-PLA, PCL-PLA multi block copolymer, PCL-polyurethane block copolymer; polyesters made from lactic, glycolic, hydroxybutyric and hydroxyl caprilic acid; polyester-anhydrides, polanhydrides, oxidized cellulose or gelatin; Poly(L-lactic acid) (L-PLA), poly(lactic-co-glycolic acid (PLGA) copolymer, polydioxanone (PDS), polyglycolide (PGA), poly(lactide-glycolide), poly(glycolic acid)/Tri-Methylene Carbonate (TMC), chitosan, cellulose, amylose, gelatin, collagen, hyaluronic acid, dextran and any derivative or mixture thereof.

50. The implant of claim 38, wherein the contrast agent is selected from the group consisting of Diatrizoic acid, Metrizoic acid, Iodamide, Iotalamic acid, Ioxitalamic acid, Ioglicic acid, Acetrizoic acid, Iocarmic acid, Methiodal, Diodone, Metrizamide, Iohexol, Ioxaglic acid, Iopamidol, Iopromide, Iotrolan, Ioversol, Iopentol, Iodixanol, Iomeprol, Iobitridol, Ioxilan, Iodoxamic acid, Iotroxic acid, Ioglycamic acid, Adipiodone, Iobenzamic acid, Iopanoic acid, Iocetamic acid, Sodium iopodate, Tyropanoic acid, Calcium iopodate, Iopydol, Propyliodone, Iofendylate, Lipiodol and non-iodinated salt, barium sulfate; Gadobenic acid, Gadobutrol, Gadodiamide, Gadofosveset, Gadolinium, Gadopentetic acid, Gadoteric acid, Gadoteridol, Gadoversetamide, Gadoxetic acid; Gadolinium oxide, carbonate, chloride, bromide fluoride, sulfates and other gadolinium salts and gadolinium complexes with organic and inorganic molecules; Iron oxide; Microspheres of human albumin, Microparticles of galactose, Perflenapent, Microspheres of phospholipids, Sulfur hexafluoride and air entrapped bubbles; and short half-life radioactive agents selected from technetium and low hazard radioactive containing tritiated molecules.

51. The implant according to claim 38, wherein the contrast agent is iron oxide and the polymer is a mixture of PLA/PCL:LA.

52. The implant according to claim 38, wherein the contrast agent is Lipiodol.

53. The implant according to claim 38, wherein the contrast agent is Lipiodol and the polymer is poly(sebacic-co-ricinoleic acid).

54. The implant according to claim 38, being in the form of a sponge.

55. The implant according to claim 54, wherein the contrast agent is Lipiodol and the polymer is PCL:LA.

56. The implant according to claim 38, wherein the contrast agent is a Gadolinium complex.

57. The implant according to claim 38, wherein the contrast agent is a Gadolinium complex and the polymer is a mixture of PLA/PCL:LA.
